(19) United States
(12) Patent Application Publication
(10) Pub. No.: US 2018/0148144 A1
(43) Pub. Date: May 31, 2018

(54) Personal Safety Device

(71) Applicant: ECOCRAFT SYSTEMS PTY LTD, Currumbin Waters, Queensland (AU)

(72) Inventor: Lee Turner, Currumbin Waters (AU)

(21) Appl. No.: 15/578,079

(22) PCT Filed: Jun. 2, 2016
(86) PCT No.: PCT/AU2016/050447

§ 371 Date: Nov. 29, 2017

(30) Foreign Application Priority Data
Jun. 2, 2015 (AU) 2015902099
Nov. 5, 2015 (AU) 2015904546
Nov. 5, 2015 (AU) 2015904548

(51) Int. Cl. B63C 9/20; G08B 25/01; A61B 5/0205; A61B 5/00; B63C 9/19

(52) U.S. Cl. CPC B63C 9/20; G08B 25/016; A61B 5/02055; A63B 24/0062; A61B 5/7282; B63C 9/19; A61B 5/4266

(57) ABSTRACT

A safety device and a method of assisting a user using the safety device. The safety device has a wearable component worn by a user or portable component able to be carried by the user, one or more sensors configured to measure the environment and/or biological attributes of a user, and a controller in communication with the one or more sensors. The controller is configured to issue a notification upon determining a predetermined condition from the one or more sensors. The issuance of the notification preferably calls for assistance and/or activates an assistance device to assist the user.

### FIELD OF THE INVENTION

The invention relates to a safety device. In particular, the invention relates, but is not limited, to a personal safety device that measures environmental and/or biological attributes and issues a notification when predetermined criteria, notably emergency criteria, are met.

### BACKGROUND TO THE INVENTION

Reference to background art herein is not to be construed as an admission that such art constitutes common general knowledge.

In various situations people can inadvertently end up in emergency situations. Whether the emergency situation is due to natural events or accidents, people often end up in a situation where assistance is required. In some cases the necessity for assistance may be the difference between life and death.

In remote environments it can be difficult to request assistance. In particularly remote environments, it may be a necessity for someone to traverse a great distance to try to obtain assistance. Modern mobile phone technology can certainly be helpful, but is reliant on there being a cellular network coverage.

Personal Locator Beacons (PLBs) and Electronic Position Indicated Radio Beacons (EPIRBs) can avoid the necessity for cellular network coverage. However, such devices require manual activation. In the event that a user is incapacitated or unaware of danger, for example they've been rendered unconscious or are trapped, it is not possible for them to trigger the PLB or EPIRB to request assistance.

### OBJECT OF THE INVENTION

It is an aim of this invention to provide a safety device which overcomes or ameliorates one or more of the disadvantages or problems described above, or which at least provides a useful alternative.

Other preferred objects of the present invention will become apparent from the following description.

### SUMMARY OF INVENTION

In one form, although it need not be the only or indeed the broadest form, there is provided a safety device comprising:

a wearable or portable component;

one or more sensors configured to measure the environment and/or biological attributes of a user;

a controller in communication with the one or more sensors;

wherein the controller is configured to issue a notification upon determining a predetermined condition from the one or more sensors.

The predetermined condition may include an environmental condition. The one or more sensors may include a water detection sensor and the condition may include a water detected condition. The one or more sensors may include a temperature sensor and the condition may include a temperature condition. The one or more sensors may include a pressure sensor and the condition may include a pressure condition. The one or more sensors may include an analyte detection sensor and the condition may include an analyte presence or property condition. The one or more sensors may include an inertial sensor and the condition may include a movement condition. The inertial sensor may include one or more accelerometers and/or a gyroscopic sensor. The one or more sensors may include a magnetic field sensor. The movement condition may include a lack of movement over a period of time. The predetermined condition may include a location, direction and/or speed condition.

The one or more sensors may include a sensor that measures biological attributes of the user. The predetermined condition may include a biological condition. The one or more sensors may include a heart rate sensor and the condition may include a heart rate condition. The one or more sensors may include a body temperature sensor and the condition may include a body temperature condition. The one or more sensors may include a breathing rate sensor and the condition may include a breathing rate condition. The one or more sensors may include a sweat sensor and the condition may include a sweat diagnosis condition. The one or more sensors may include a blood oxygen sensor and the condition may include a blood oxygen condition. The one or more sensors may include a respiration sensor. The respiration sensor may measure a chest girth measurement sensor. The chest girth measurement sensor is preferably a variable inductance based sensor.

The one or more sensors may include a location sensor that calculates a position. The one or more sensors may include a sensor that measures altitude. The one or more sensors may include a Global Navigation Satellite System (GNSS) sensor (e.g. a GPS and/or GLONASS receiver). The condition may include a location condition. The sensor may comprise a radio signal sensor. The radio signal sensor may include a mobile telephone network sensor. The mobile telephone network sensor may include a SIM card to access a mobile phone network.

It will be appreciated that a plurality of sensors may be utilised to determine the predetermined condition. The predetermined condition may include a plurality of sub-conditions. There may be a plurality of predetermined conditions.

Determination of the predetermined condition may comprise a time component. The time component may be a time delay or a minimum time period. Determination of the predetermined condition may comprise measuring time over which a measurement from the one or more sensors is at a predetermined level, or within a predetermined range, and determining the condition if the measurement from the one or more sensors is at a predetermined level, or within a predetermined range, exceeds a predetermined minimum time period. Preferably the predetermined condition considers patterns of measurements from the one or more sensors over time.

Preferably the safety device is a personal safety device. Preferably the predetermined condition is considered an emergency condition. Preferably the personal safety device comprises a signal that advises a user when the predetermined condition is about to be met. Preferably the personal safety device comprises a signal that advises a user when the predetermined condition has been met. The signal may comprise an audio, visual, and/or haptic signal. The personal safety device may further comprise a deactivation input to allow a user to prevent or halt issuance of the notification. The personal safety device may further comprise an activation input to allow a user to manually issue a notification.

In another form the safety device may be attached to a vehicle. Preferably the vehicle is a watercraft. Preferably the safety device has a mount to affix the safety device to the vehicle.

Preferably the wearable component comprises a harness. Preferably the wearable component comprises one or more straps with one or more fasteners. The wearable component may comprise an article of clothing. The wearable component preferably comprises an appendage restraint and enclosure for retaining the device against the user's appendage.

In one form the controller is in communication with a wireless communication system. Preferably the issuance of a notification from the controller activates the wireless communication system. The communication system may comprise satellite communication. The communication system may comprise radio communication. The communication system preferably transmits a message or signal. The message may comprise information from the one or more sensors. The message may comprise information regarding the determined condition. Preferably, the message at least comprises information regarding the location of the device. Preferably the safety device includes a wireless communication system. In another form the safety device may utilise an external wireless communication system via a suitable interface. The communication system may comprise a polling system. The polling system preferably polls the external wireless communication system.

The personal safety device may comprise an assistance device which is activated or deployed in response to the notification. The assistance device may comprise, for example, a buoyance device, nutrition, medication, one or more tools, a light source, or the like.

Preferably the personal safety device is in wireless communication with a receiver of the assistance device. Preferably the personal safety device is in communication with the assistance device wirelessly using, for example, near field communication (NFC). The personal safety device may be connected, mounted, or affixed to the assistance device, and/or may be contained within at least a portion of the assistance device.

In one form the safety device may comprise a self inflating personal safety device having a buoyance device that is triggered to inflate upon receiving the notification from the controller. The buoyancy device preferably comprises an inflatable member. Preferably the inflatable member is an inflatable bladder. Preferably the inflatable member is in fluid communication with a propellant. Preferably the propellant comprises compressed gas. Preferably the compressed gas is contained in a cartridge. The compressed gas may comprise carbon dioxide, nitrogen, nitrous oxide, or the like. In another form the propellant may comprise chemical reactants.

In one form, the notification system may comprise an alarm. The alarm may comprise a light output. The light output may comprise an LED. The light output may comprise a laser. The alarm may comprise an audible output. The audible output is preferably a siren. The siren may comprise a piezoelectric mechanism. The alarm may be in wired or wireless communication with the controller. The alarm may comprise its own power supply.

Preferably the controller is electronic. Preferably the personal safety device further comprises a power system comprising a power source in electrical connectivity with the controller. Preferably the controller comprises a microcontroller. In an alternative form the controller may comprise a microprocessor. Preferably the microcontroller or microprocessor comprises a plurality of inputs and outputs. Preferably at least one sensor is in communication with at least one input.

Preferably the personal safety device comprises a housing. Preferably the housing is sealed. Preferably the housing is waterproof. Preferably at least the controller is contained within the housing. Preferably the power system is also contained within the housing. At least a portion of the one or more sensors may be contained within the housing. At least a portion of the one or more sensors may be located outside the housing. Preferably any sensors located outside the housing are in wireless communication with the controller. In a preferred form they are in wireless communication using NFC.

The safety device may further comprise a data log. The data log preferably stores previous sensor measurements. The data log preferably has a data interface to allow at least a portion of the data log to be retrieved. Preferably the data log comprises memory. Preferably the memory is non-volatile. Preferably the data log is configured to store information derived from the one or more sensors. Preferably the data log is configured to store information derived from more than one measurement of the one or more sensors over a period of time. Preferably the data log stores a record of sensor measurements from before an emergency for later analysis.

In another form, there is provided a method of assisting a user having a safety device, the method comprising the steps of:

measuring environmental and/or biological attributes of a user of the safety device using one or more sensors;

communicating measurements from the one or more sensors to a controller;

determining, at the controller, whether a predetermined condition has occurred using the measurements communicated from the one or more sensors; and

issuing a notification upon determining that the predetermined condition has occurred.

Preferably the safety device is a safety device as hereinbefore described. The step of determining, at the controller, whether a predetermined condition has occurred preferably comprises considering the measurements over a predetermined time period. Preferably the predetermined condition is considered an emergency condition. Preferably there are a plurality of predetermined conditions and the controller determines whether any one (or more) of the predetermined conditions has occurred.

The method may further comprise the step of activating an alarm. The step of activating the alarm is preferably in response to a predetermined condition being considered imminent. In another form the alarm may be activated in response to the notification. The method may further comprise activating an assistance device in response to the notification. The method may further comprise communicating a message in response to the notification. The method preferably comprises a combination of activating an alarm, activating an assistance device, and communicating a message in response to the notification.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 illustrates a high level functional view;

FIG. 2 illustrates a high level schematic view of an example device;

FIG. 3 illustrates a first user using a safety device in a first example situation;

FIG. 4 illustrates a second user using the safety device in a second example situation;

FIG. 5 illustrates the safety device of FIG. 4 in a deployed state when in use; and

FIG. 6 illustrates the safety device of FIG. 4 in a deployed state by itself.

### DETAILED DESCRIPTION OF THE DRAWINGS

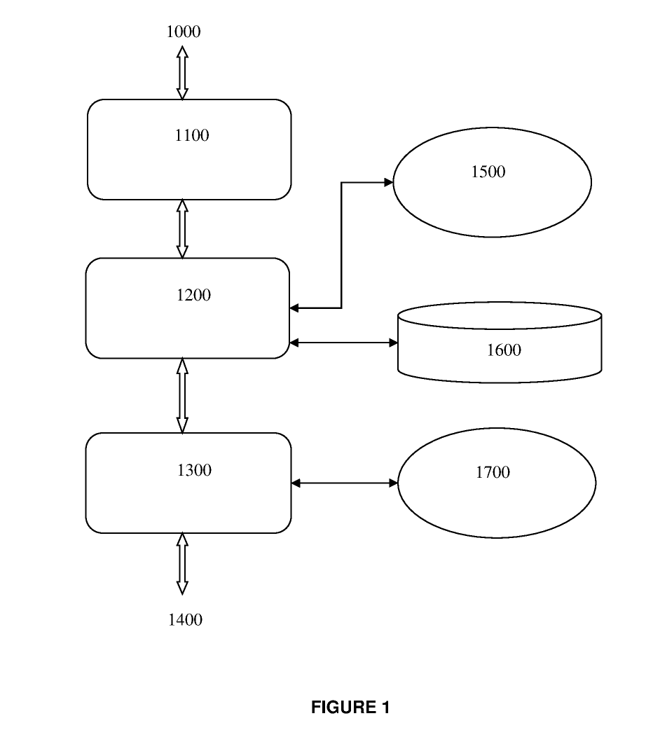

FIG. 1 illustrates a high level functional view of a safety device where data 1000 is collected by sensors 1100 and transmitted to a controller 1200. The controller 1200 utilises information from the sensors 1100 for pattern recognition and control. The collected data and optionally its history can be considered to enable the controller 1200 to warn a user of one or more specific situations and/or to activate one or more assistance devices 1300 implemented to try to address a situation 1400.

Preferably, a user interface 1500 is provided to allow user to influence the actions of the controller 1200. Preferably, a data log 1600 is provided to collect data for research or analysis. For example, to assist the controller 1200 in determining whether a predetermined condition has occurred, or in retrospectively determining how a dangerous situation developed.

When appropriate, the controller activates one or more assistance devices 1300 to try to assist the user in addressing the situation. Preferably, a manual override 1700 is provided to override the actions of the controller 1200. For example, the manual override may be utilised to manually activate the one or more assistance devices 1300, or may be utilised to prevent the controller 1200 from activating the one or more assistance devices 1300.

In a water sports example, the safety device may comprise two sensors 1100, pressure and acceleration, and if the two sensors indicate to the controller 1200 that a user has been underwater for a predefined number of seconds and follows a distinct movement pattern the controller 1300 might alert the user. If no reaction to the warning is encountered, indicating that the user is possibly unconscious, the controller 1300 sends a signal to activate the assistance device 1300, such as an inflatable bladder, and activate a siren to assist the user and/or rescuers.

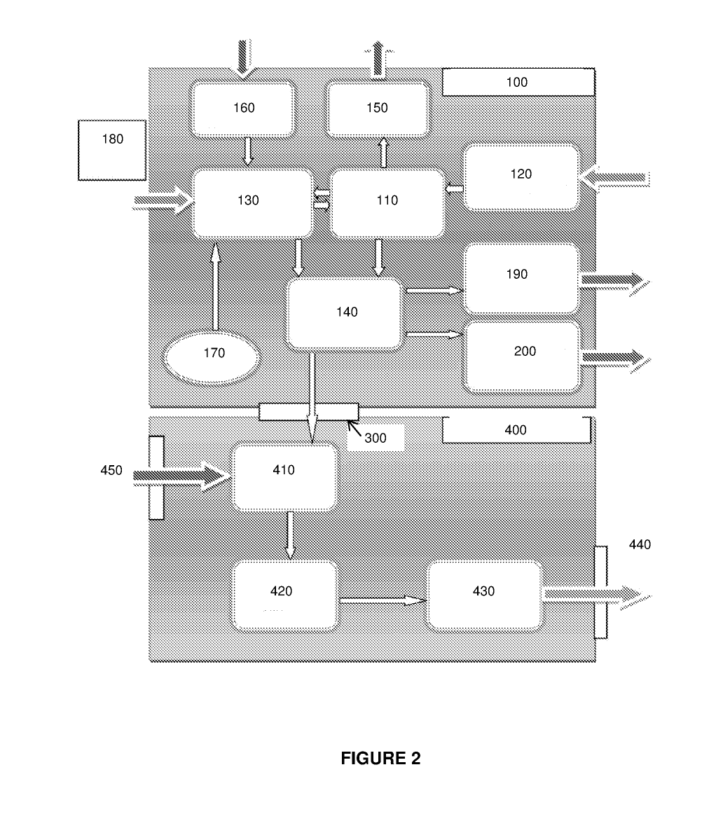

FIG. 2 illustrates a high level schematic of a safety device having a control module 100 and a communication module 400. The control module 100 is connected to the communication module 400 via connection 300. The connection 300 may be a physical wired connection, or may be a wireless connection.

The control module 100 has a microcontroller 110 in communication with sensors 120 such as, for example, environmental sensors, biological sensors, and/or location sensors. The control module 100 may be in wired or wireless communication with the sensors 120. In a preferred form the communication module 100 is in wireless communication with at least some sensors 120 via near field communication (NFC).

The microcontroller 110 is connected to power system 130, a notification module 140, and an indicator LED 150. A test switch 160 is connected to the power system 130 which in turn is connected to a power source in the form of a battery 170. The power system may also be connected to a power switch 180 which may be automated to actuate in response to a measurement from the sensors 120. The notification system is connected to a visual light output 190 and an audible output 200 configured to alert a user or nearby people.

A communication interface 410 of the communication module 400 is configured to receive notifications from the notification system 140 of the controller module 100. The communication interface 410 is also connected to a manual interface 450, e.g. an external switch or cord, which can be used for manual control such as activation or deactivation of the communication module 400 without a notification from the notification system 140. The communication interface 410 is configured to output a message 420 to a transmitter 430 which in turn emits a wireless signal 440 which is typically a radio signal.

In some forms, the safety device 10 may have a different, or additional, component to the communication module 400. In such forms, the communication module 400 may be replaced, or complemented, by an assistance device which is activated or deployed in response to the notification.

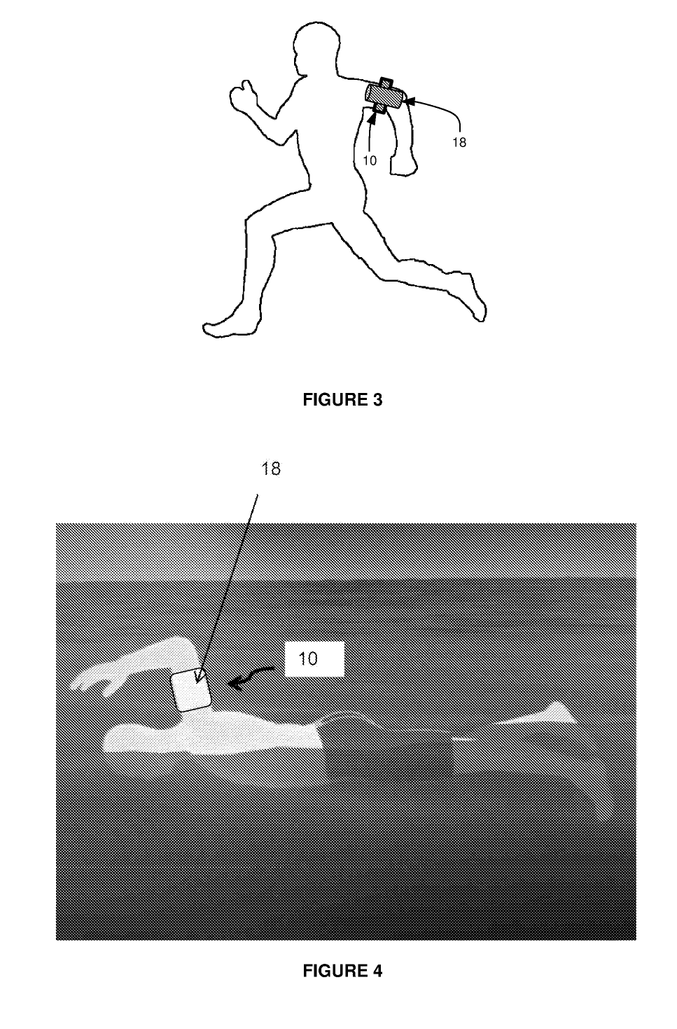

Examples of a safety device 10 in use are illustrated in FIGS. 2 and 3. The safety device 10 has a wearable component which, in both FIGS. 2 and 3, is in the form of a cuff or armband 18. No express limitation is meant thereby and it will be appreciated that the safety device could be worn on different appendages, or could be integral with other clothing or equipment (e.g. a safety hat). Furthermore, the example illustrated in FIG. 3 is in use on a running user and the example illustrated in FIG. 4 is in use on a swimmer but, again, no express limitation is meant thereby and it will be appreciated that the safety device 10 could be used in relation to a large number of different applications including, for example, combat, sports, hiking, working in remote locations or dangerous areas, or the like.

The safety device 10 has sensors 120 configured to measure environment and/or biological attributes of a user. For example, the sensors may measure environmental and/or user temperature, pressure, the presence of water, heart rate, breathing rate, sweat, bloody oxygen, analytes, or the like. Location sensors are also preferably provided, preferably in the form of GNSS received configured to provide a GNSS position of the safety device 10. It will be appreciated that the sensors will be chosen to match a particular application for the safety device. For example, for the running user illustrated in FIG. 3 the sensors may include a heart rate and location sensors and for the swimming user in FIG. 4 the sensors may include a water pressure sensor.

The microcontroller 110 is configured to sample the sensors, continually, periodically, or on demand as determined is necessary by the microcontroller 110, and determines whether a predetermined condition has been met. The predetermined condition will typically be when a sensor measurement is outside one or more predetermined ranges. The microcontroller 110 typically also considers a time period, such that brief measurements outside the one or more predetermined ranges do not necessarily result in the predetermined condition being met, but rather measurements outside the predetermined range for a predetermined minimum time period may result in the predetermined condition being met.

Once a predetermined condition is detected, which is typically a condition considered to be an emergency condition, the microcontroller 110 signals the notification system 140 which issues a notification. The form of the notification, and what it triggers, may vary depending on the application.

For example, for the configuration illustrated in FIG. 2 which would likely be suitable for the running user illustrated in FIG. 3, the notification is transmitted to the communication module 400. The communication module 400 then prepares a message 420 which is transmitted by transmitter 430 as wireless signal 440.

In the running user application illustrated in FIG. 3 the sensors 120 may include a heart rate sensor which determines a physical state of the user. The predetermined condition may be that the heart rate is outside a predetermined ‘safe’ range for a period of time. Upon that predetermined condition being met, the micro controller 110 signals the notification system 140 to issue a notification which activates the communication module 400 to send a wireless signal 440. In this particular embodiment the sensors 120 preferably also include a location sensor and the message 420 preferably includes the location of the user to enable assistance to locate and assist the user.

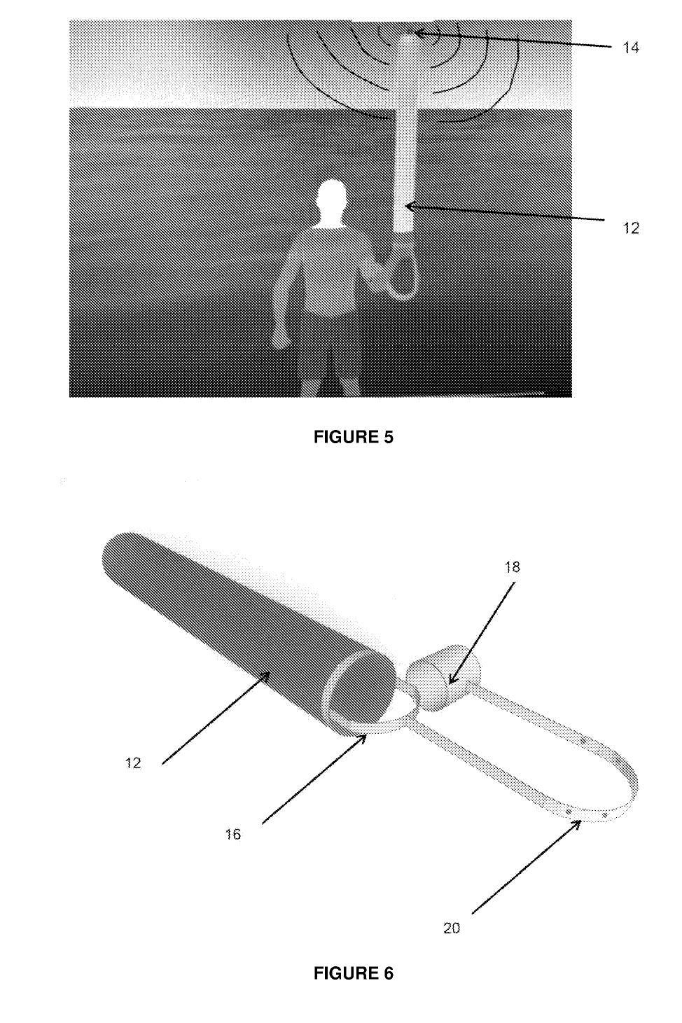

In the swimming user application illustrated in FIG. 3, the sensors 120 may include a pressure sensor which determines the depth of the user. The predetermined condition may be that the pressure is over a certain value for a period of time. Upon that predetermined condition being met, the micro controller 110 signals the notification system 140 to issue a notification. This could activate a communication module 400 as per the previous application. Instead, or additionally, the notification may be used to activate an assistance device such as, for example, a self inflating inflatable tube 12, as seen in FIGS. 5 and 6.

As illustrated in FIG. 4 the safety device 10 is in a non-deployed state. As illustrated in FIG. 5 the safety device 10 is in a deployed state. As shown in FIG. 4, at a distal end (relative to the user) is an alarm having a light output in the form of a safety light 14. In a form, when inflated the inflatable tube 12 also has an audible output, preferably in the form of a 120 db+ electronic siren (not shown). The siren and safety light 14 are typically activated upon inflation of the inflation tube 12.

Upon receiving a notification from the notification system 140, an actuator inflates the inflatable tube 12 with propellant which is preferably compressed gas stored within a compressed gas cartridge (not shown). As seen most clearly in FIG. 6, the cuff 18 is connected to the inflation tube 12 via tether 20. The inflation tube 12 also has at its bottom end (proximal end relative to the user) a handle 16 which joins both the inflation tube 12 and the tether 20. The handle 16 may be used by a user to handle the inflation tube 12 and remain afloat. By placing weight on the handle 16, the inflation tube extends vertically out of the water as shown in FIG. 5.

The running user (FIG. 3) and swimming user (FIG. 4) are for example only and different configurations could be utilised depending on the intended application. The microcontroller 110 may be programmed to detect a number of different predetermined conditions to issue a notification. Generally, however, the safety device 10 is configured to detect a predetermined condition, preferably aligning with an emergency situation, and issues a notification which, in one form or another, preferably assists the user. The assistance may be in the form of notifying others of the need for assistance (e.g. via wireless signal 440), and/or it may be in the form of providing the user with the necessary assistance (e.g. as shown in FIGS. 3 to 4).

In either application the need for the predetermined condition to be determined by the microcontroller 140 may be bypassed with the manual interface 450. The manual interface 450 may also be used to deactivate or temporarily suppress the safety device 10 should a predetermined condition be met but the user wants to prevent or halt issuance of the notification.

The microcontroller 110 may be configured to measure the sensors 120 at given intervals and checks for predetermined conditions which may comprise particular patterns. If no pattern is recognised the microcontroller 110 continues to sample the sensors 120. A motion sensor may detect movement and, if (1) no movement is detected for a prescribed period of time and (2) the sensors 120 measure a safe situation, then the microcontroller 110 may go into a low power ‘sleep’ state. In a form the microcontroller 110 may operate in a sleep state continuously, with only a watchdog timer running to activate the next measurement and operate the pattern matching algorithm for the predetermined condition. The test switch 160 may be used to initiate a system check and indicate system status with a signal from an LED and/or buzzer.

Advantageously, the safety device 10 allows for an automatic notification to be issued upon determined predetermined conditions of a user and/or their environment. Accordingly, even if a user is incapacitated, either because they are trapped or rendered unconscious, it may be possible for assistance to be sought automatically. Information regarding the user, including their condition and/or location, can be communicated via a communication module 400. This not only allows for help to be called automatically in the event of an emergency, but also enables the assisting party to be given important information that may be able to assist to user.

Through careful selection of sensors and predetermined conditions, the device is able to ‘intelligently’ issue notifications only when significant situations arise. False positives can be minimised greatly by relying in information from a plurality of sensors to ascertain the likely situation and whether assistance is required. For example, if a user starts moving quickly and their heart rate increases significantly this may not be cause for concern. On the other hand, if a user is stationary and their heart rate increases significantly then this may be cause for concern. The predetermined conditions therefore preferably considers measurements from an array of sensors in a holistic manner. This results in an ‘intelligent’ system which can accurately ascertain a situation and react accordingly without user intervention.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step etc.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

1. A safety device comprising:
a wearable or portable component;
one or more sensors configured to measure one or more of the environment and biological attributes of a user;
a controller in communication with the one or more sensors;
wherein the controller is configured to issue a notification upon determining a predetermined condition from the one or more sensors.

2. The safety device of claim 1, wherein the predetermined condition includes an environmental condition.

3. The safety device of claim 2, wherein the one or more sensors include one or more of: water detection sensor, a temperature sensor, a pressure sensor, an analyte detection sensor, an inertial sensor, and a magnetic field sensor.

4. The safety device of claim 2, wherein the predetermined condition includes one or more of: a water detected condition, a temperature condition, a pressure condition, an analyte presence or property condition, a movement condition, and a location, direction and/or speed condition.

5. The safety device of claim 1, wherein the one or more sensors include a sensor that measures biological attributes of the user.

6. The safety device of claim 5, wherein the one or more sensors include one or more of: a heart rate sensor, a body temperature sensor, a breathing rate sensor, a sweat sensor, a blood oxygen sensor, and a respiration sensor.

7. The safety device of claim 5, wherein the predetermined condition includes one or more of: a heart rate condition, a body temperature condition, a breathing rate condition, a sweat diagnosis condition, a blood oxygen condition, a respiratory condition.

8. The safety device of claim 1, wherein the one or more sensors includes a location sensor that calculates a position.

9. The safety device of claim 8, wherein the one or more sensors includes one or more of: an attitude sensor, a Global Navigation Satellite System (GNSS) sensor and a radio signal sensor.

10. The safety device of claim 8, wherein the predetermined condition includes a location condition.

11. The safety device of claim 1, wherein a plurality of sensors are utilised to determine the predetermined condition.

12. The safety device of claim 11, wherein the predetermined condition includes a plurality of sub-conditions.

13. The safety device of claim 1, wherein the predetermined condition comprises a time component.

14. The safety device of claim 13, wherein the time component comprises a time delay or a minimum time period.

15. The safety device of claim 14, wherein determination of the predetermined condition comprises measuring time over which a measurement from the one or more sensors is at a predetermined level, or within a predetermined range, and determining the condition if the measurement from the one or more sensors is at a predetermined level, or within a predetermined range, exceeding a predetermined minimum time period.

16. The safety device of claim 1, wherein the predetermined condition is altered to take into account patterns of measurements from the one or more sensors over time.

17. The safety device of claim 1, wherein the controller is configured to issue a signal that advises the user when the predetermined condition is about to be met.

18. The safety device of claim 17, wherein the signal comprise an audio, visual, and/or haptic signal.

19. The safety device of claim 1, further comprising a deactivation input to allow the user to prevent or halt issuance of the notification.

20-31. (canceled)

32. A method of assisting a user having a safety device, the safety device comprising a wearable or portable component; one or more sensors configured to measure one or more of the environment and biological attributes of a user; a controller in communication with the one or more sensors; wherein the controller is configured to issue a notification upon determining a predetermined condition from the one or more sensors, the method comprising the steps of:
measuring one or more of environmental attributes and biological attributes of a user of the safety device using one or more sensors;
communicating measurements from the one or more sensors to a controller;
determining, at the controller, whether a predetermined condition has occurred using the measurements communicated from the one or more sensors; and
issuing a notification upon determining that the predetermined condition has occurred.

33-39. (canceled)
